(19) United States
(12) Patent Application Publication
(10) Pub. No.: US 2018/0311199 A1
(43) Pub. Date: Nov. 1, 2018

(54) COMBINATION TREATMENT FOR INFLAMMATORY DISEASES

(71) Applicant: Lawrence CHAN, Chicago, IL (US)

(72) Inventor: Lawrence CHAN, Chicago, IL (US)

(21) Appl. No.: 15/502,318

(22) PCT Filed: Jan. 11, 2017
(86) PCT No.: PCT/US2017/013069

§ 371 Date: Feb. 7, 2017

Related U.S. Application Data
(60) Provisional application No. 62/277,489, filed on Jan. 12, 2016.

(51) Int. Cl. A61K 31/222; A61P 17/06; A61P 19/02; A61P 29/00; C07K 16/24

(52) U.S. Cl. CPC A61K 31/222; A61P 17/06; A61P 19/02; A61K 39/3955; C07K 16/241; A61K 2300/00; A61P 29/00

(57) ABSTRACT

The present disclosure relates to a combination treatment for inflammatory diseases. Specifically, the treatment described herein involves administering to a subject suffering from an inflammatory disease a combination of an anti-TNF-alpha inhibitor and at least one small molecule anti-inflammatory agent, to treat the inflammatory disease.

### CROSS-REFERENCE TO RELATED APPLICATIONS

This application claims priority to U.S. application Ser. No. 62/277,489 filed on Jan. 12, 2016, the contents of which are herein incorporated by reference.

### FIELD OF THE INVENTION

The present disclosure relates to a combination treatment for inflammatory diseases. More specifically, the treatment described herein involves administering to a subject suffering from an inflammatory disease a combination of a TNF-alpha inhibitor and at least one small molecule anti-inflammatory agent, either simultaneously or sequentially, to treat the inflammatory disease.

### BACKGROUND OF THE INVENTION

Chronic inflammatory diseases, such as psoriasis, are characterized by tissue infiltration of inflammatory cells and dysregulation (increase or decrease) natural chemicals called cytokines and chemokines that promote and sustain the inflammation. Treatments of inflammatory diseases naturally focus on discovery and development of medications that produce inhibition of these inflammatory components, i.e., anti-inflammatory drugs. Currently, biologics, such as inhibitor of a natural chemical called tumor necrosis factor (anti-TNF-alpha), have been used widely for treatment of inflammatory diseases such as psoriasis and rheumatoid arthritis and are generally used as a mono-therapeutic agent.

Monotherapeutic biologics such as anti-TNF-alpha commonly contain partial animal proteins which eventually induce the human body to generate antibodies against it. As a result, such biologics face the possibility of loss efficacy during the course of treatment. Moreover, treatments employing biologics typically rely on the ability of these medications to bind to the specific target site in a three-dimensional conformation fashion. Such binding could be weakened by certain physiological conditions such as temperature and pH. Additionally, the cost of biologic medications are extremely high and putting a tremendous burden on payor systems throughout the world. As a result, there is a need in the art for alternative treatment regimens to those that solely employ the use of monotherapeutic agents that are highly efficacious, are cost effective and are stable.

### BRIEF SUMMARY OF THE INVENTION

The present disclosure relates to method of treating a subject suffering from an inflammatory disease, such as chronic or acute inflammatory disease. Specifically, the method involves administering to the subject in need of treatment (such as suffering from a chronic or an acute inflammatory disease) thereof a therapeutically effective amount of at least one anti-TNF alpha inhibitor and a therapeutically effective amount diacerein to treat the inflammatory disease. An anti-TNF alpha inhibitor used in the methods described herein can be infliximab, adalimumab, certolizumab, etanercept, golimumab or combinations thereof. In certain embodiments, the anti-TNF alpha inhibitor is infliximab. In certain embodiments, the anti-TNF alpha inhibitor and diacerein are administered simultaneously. In certain other embodiments, the anti-TNF alpha inhibitor and diacerin are administered sequentially.

In certain embodiments, the amount of anti-TNF alpha inhibitor administered to the subject is about 0.25 mg/kg to about 10 mg/kg. In certain embodiments, the amount of diacerein administered to the subject is about 10 mg to about 75 mg. The diacerein can be administered to the subject once or twice daily.

In certain embodiments, the chronic inflammatory disease to be treated pursuant to the method described herein is rheumatoid arthritis, psoriasis, psoriatic arthritis, non-infectious uveitis, ulcerative colitis, Crohn's disease, ankylosing spindilitis, hidradenitis suppurativa or lichen planus.

### BRIEF DESCRIPTION OF THE DRAWINGS

FIGS. 1a-e show a group of bar graphs of proteins (IL-6, IL-19, CSF-3, S100A8, NAP-2, respectively) as assayed by ELISA method described in Example 1. Legends: Cont, control culture medium; M5, culture incubated with 5 pro-inflammatory cytokines that recapitulate the in vivo immune milieu of lesional psoriasis; M5+D10, culture incubated with M5 and diacerein (10 μM); M5+R5, culture incubated with M5 and Remicade (5 μg/ml); M5+D10+R5, culture incubated with M5 plus diacerein (10 μM) and Remicade (5 μg/ml).

FIG. 2 shows a bar graph of mRNAs for IL-8, as assayed by reverse transcription followed by real-time PCR methods as described in Example 1. Legends: Cont, control culture medium; M5, culture incubated with 5 pro-inflammatory cytokines that recapitulate the in vivo immune milieu of lesional psoriasis; Diac, culture incubated with M5 and diacerein (10 μM); Remicade, culture incubated with M5 and infliximab (Remicade®) (5 μg/ml); Diac+Rem, culture incubated with M5 plus diacerein (10 μM) and infliximab (5 μg/ml).

FIG. 3 shows a bar graph of mRNAs for ICAM-1 as assayed by reverse transcription followed by real-time PCR methods as described in Example 1. Legends: Cont, control culture medium; M5, culture incubated with 5 pro-inflammatory cytokines that recapitulate the in vivo immune milieu of lesional psoriasis; Diac, culture incubated with M5 and diacerein (10 μM); infliximab, culture incubated with M5 and infliximab (5 μg/ml); Diac+Rem, culture incubated with M5 plus diacerein (10 μM) and infliximab (5 μg/ml).

### DETAILED DESCRIPTION OF THE INVENTION

### Introduction

The present disclosure relates to a unique combination treatment for inflammatory diseases. The treatment described in more detail involves administering to a subject suffering from an inflammatory disease (such as a chronic or acute inflammatory disease) a combination of an anti-TNF-alpha inhibitor and diacerein, a small molecule anti-inflammatory agent, to treat the inflammatory disease. It was discovered that administering a combination anti-TNF-alpha inhibitor and diacerein results in a synergistic therapeutic effect that provides a superior treatment option for patients suffering from inflammatory diseases, particularly chronic inflammatory diseases.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass.

“Anti-TNF alpha” (anti-TNF-α) or “anti-TNF alpha inhibitor” (anti-TNF-α inhibitor) as used herein interchangeably, refers to an antibody that can be administered to a subject to moderate or suppress a physiologic response to tumor necrosis factor (TNF), such as in a subject suffering from an inflammatory disease. Examples of anti-TNF alpha inhibitors include infliximab, adalimumab, certolizumab, etanercept, golimumab or combinations thereof.

“Effective amount”, “pharmaceutically effective amount”, and “therapeutically effective amount” as used interchangeably herein refer to an amount that may be effective to elicit the desired biological or medical response, including the amount of a compound, agent and/or antibody that, when administered to a subject for treating a disease, is sufficient to effect such treatment for the disease. The effective amount will vary depending on the compound, the disease and its severity and the age, weight, etc., of the subject to be treated. The effective amount can include a range of amounts. A pharmaceutically effective amount includes amounts of a compound, agent and/or antibody, which are effective when combined with other agents.

“Inflammatory diseases” or “inflammatory disorders” as used herein, refer to diseases wherein the typical characteristics of inflammation are observed. This term can therefore overlap with other diseases wherein an inflammation aspect is also present. It is known in the art that a distinction can be made between “acute inflammation” and “chronic inflammatory diseases”. The term “inflammatory diseases” or “inflammatory disorders” includes but is not limited to diseases such as rheumatoid arthritis, conjunctivitis, rheumatoid spondylitis, osteoarthritis, gouty arthritis, bronchitis, tuberculosis, chronic cholecystitis, acute pancreatitis, sepsis, asthma, chronic obstructive pulmonary disease, ankylosing spindilitis, hindradentis suppurativa, lichen planus, psoriatic arthritis, non-infectious uveitis, erythema multiforme, Stevens-Johnson syndrome, dermal inflammatory disorders such as psoriasis, atopic dermatitis, and toxic epidermal necrosis, systemic inflammatory response syndrome (SIRS), acute respiratory distress syndrome (ARDS), cancer-associated inflammation, reduction of tumor-associated angiogenesis, diabetes, graft versus host disease and associated tissue rejection inflammatory responses, inflammatory bowel diseases such as ulcerative colitis and Crohn's disease, a delayed-type hypersensitivity, immune-mediated and inflammatory elements of CNS disease; e.g., Alzheimer disease, Parkinson disease, multiple sclerosis, etc.

In certain embodiments, the chronic inflammatory disease is psoriasis. In certain embodiments, the chronic inflammatory disease is rheumatoid arthritis. In certain embodiments, the chronic inflammatory disease is an inflammatory bowel disease such as ulcerative colitis and Crohn's disease. In certain embodiments, the chronic inflammatory is ankylosing spindilitis. In certain embodiments, the chronic inflammatory disease is psoriatic arthritis. In certain embodiments, the chronic inflammatory disease is non-infectious uveitis. In certain embodiments, the chronic inflammatory disease is hidradenitis suppurativa. In certain embodiments, the chronic inflammatory disease is lichen planus. In certain embodiments the chronic inflammatory disease is rheumatoid arthritis, psoriasis, ulcerative colitis, Crohn's disease, psoriatic arthritis, non-infectious uveitis, ankylosing spindilitis, hidradenitis suppurativa or lichen planus. In certain embodiments, the acute inflammatory disease is erythema multiforme. In certain embodiments, the acute inflammatory disease is Stevens-Johnson syndrome. In certain embodiments, the acute inflammatory disease is toxic epidermal necrosis.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

The terms “treatment” or “treating” as used herein refer to an approach for obtaining beneficial or desired results including clinical results. Beneficial or desired clinical results may include one or more of the following: (i) decreasing one more symptoms resulting from the disease; (ii) diminishing the extent of the disease and/or stabilizing the disease (e.g., delaying the worsening of the disease); (iii) delaying the spread of the disease; (iv) delaying or slowing the onset or recurrence of the disease and/or the progression of the disease; (v) ameliorating the disease state and/or providing a remission (whether partial or total) of the disease and/or decreasing the dose of one or more other medications required to treat the disease; (vi) increasing the quality of life, and/or (vii) prolonging survival.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### Combination Treatment

The present disclosure provides a unique combination treatment for subjects suffering from inflammatory diseases. In certain embodiments, the present disclosure provides a combination treatment for treating subjects suffering from chronic inflammatory diseases. Specifically, the treatment involves administering to a subject suffering from an inflammatory disease a combination of a therapeutically effective amount of at least one anti-TNF-alpha inhibitor and a therapeutically effective amount at least one anti-inflammatory agent. Specifically, synergistic results were discovered when the anti-inflammatory agent diacerein was used in the methods described herein. These synergistic results provide a superior treatment option for patients suffering from inflammatory diseases, particularly, chronic inflammatory diseases.

In the methods described herein, any anti-TNF-alpha inhibitor can be used. In certain embodiments, the anti-TNF-alpha inhibitor is infliximab, adalimumab, certolizumab, etanercept, golimumab or combinations thereof. In certain embodiments, the anti-TNF-alpha inhibitor is infliximab. In certain embodiments, the anti-TNF-alpha inhibitor is adalimumab. In certain embodiments, the anti-TNF-alpha inhibitor is certolizumab. In certain embodiments, the anti-TNF-alpha inhibitor is etanercept. In certain embodiments, the anti-TNF-alpha inhibitor is golimumab.

The anti-TNF-alpha inhibitor can be administered to the subject using any technique known in the art. For example, the anti-TNF-alpha inhibitor can be administered orally, intravenously, subcutaneously or using any combinations thereof.

The amount of anti-TNF-alpha inhibitor to be administered to a subject can be determined using routine techniques known in the art depending on the anti-TNF-alpha inhibitor to be used and the inflammatory disease to be treated. Generally, with anti-TNF-alpha inhibitors, the subject receives one or more loading doses followed by daily, weekly or monthly maintenances doses to achieve and maintain a target, therapeutically effective serum level of the antibody. In certain embodiments, the subject will receive from about 0.25 mg/kg to about 10 mg/kg maintenance doses of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 0.25 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 0.5 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 1 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 2 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 3 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 4 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 5 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 6 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 7 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 8 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 9 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated. In certain embodiments, the subject will receive from about 10 mg/kg of the anti-TNF-alpha inhibitor, daily, every other day, every week, every two weeks, every three weeks, every four weeks, every five weeks, every six weeks, every seven weeks, every eight weeks, every nine weeks, every ten weeks, every eleven weeks or every twelve weeks depending on the inflammatory disease to be treated.

In certain other embodiments, the subject is administered a therapeutically effective amount of anti-TNF-alpha inhibitor to achieve a target serum level of between 0.2 μg/ml and 40 μg/ml. In certain embodiments, the subject is administered a therapeutically effective amount of an anti-TNF-alpha inhibitor to achieve a target serum level of about 0.2 μg/ml, 0.5 μg/ml, 1.0 μg/ml, 2.0 μg/ml, 3.0 μg/ml, 4.0 μg/ml, 5.0 μg/ml, 6.0 μg/ml, 7.0 μg/ml, 8.0 μg/ml, 9.0 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 or 40 μg/ml. In certain embodiments, the subject is administered a therapeutically effective amount of infliximab to achieve a target serum level of about 0.2 μg/ml, 0.5 μg/ml, 1.0 μg/ml, 2.0 μg/ml, 3.0 μg/ml, 4.0 μg/ml, 5.0 μg/ml, 6.0 μg/ml, 7.0 μg/ml, 8.0 μg/ml, 9.0 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 or 40 μg/ml. In certain embodiments, the subject is administered a therapeutically effective amount of adalimumab to achieve a target serum level of about 0.2 μg/ml, 0.5 μg/ml, 1.0 μg/ml, 2.0 μg/ml, 3.0 μg/ml, 4.0 μg/ml, 5.0 μg/ml, 6.0 μg/ml, 7.0 μg/ml, 8.0 μg/ml, 9.0 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 or 40 μg/ml. In certain embodiments, the subject is administered a therapeutically effective amount of certolizumab to achieve a target serum level of about 5 μg/ml, 10 μg/ml, 20 μg/ml, 30 μg/ml, or 40 μg/ml. In certain embodiments, the subject is administered a therapeutically effective amount of etanercept to achieve a target serum level of about 0.2 μg/ml, 0.5 μg/ml, 1.0 μg/ml, 2.0 μg/ml, 3.0 μg/ml, 4.0 μg/ml, 5.0 μg/ml, 6.0 μg/ml, 7.0 μg/ml, 8.0 μg/ml, 9.0 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 or 40 μg/ml. In certain embodiments, the subject is administered a therapeutically effective amount of golimumab to achieve a target serum level of about 0.2 μg/ml, 0.5 μg/ml, 1.0 μg/ml, 2.0 μg/ml, 3.0 μg/ml, 4.0 μg/ml, 5.0 μg/ml, 6.0 μg/ml, 7.0 μg/ml, 8.0 μg/ml, 9.0 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 or 40 μg/ml.

In the methods described herein, a therapeutically effective amount of the anti-inflammatory agent diacerein is administered with the therapeutically effective amount of at least one anti-TNF-alpha inhibitor. The diacerein and the anti-TNF-alpha inhibitor can be administered either simultaneously or sequentially, in any order. The order is not critical. The diacerein can be administered to a subject using any technique known in the art such as orally, intravenously, subcutaneously or any combinations thereof. Additionally, the amount of diacerein to be administered to a subject can be determined using routine techniques known in the art. For example, a subject can be administered 10 mg, 25 mg, 50 mg, or 75 mg of diacerein once or twice daily in order to obtain a target serum level of between about 0.2 μM and about 15 μM. In certain embodiments, the subject is administered 50 mg of diacerein once or twice daily in order to obtain a serum level of about 0.2 μM, about 0.5 μM, about 1 μM, about 2 μM, about 3 μM, about 4 μM, about 5 μM, about 6 μM, about 7 μM, about 8 μM, about 9 μM, 10 μM, 11 μM, 12 μM, 13 μM, 14 μM or about 15 μM.

### EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present invention has multiple aspects, illustrated by the following non-limiting example.

### Example 1

Experiments were carried out to investigate the effect of combined anti-TNF-α (infliximab) and diacerein, an inhibitor for a pro-inflammatory cytokine interleukin-1, utilizing an in vivo-equivalent psoriasis cell culture system. This in vivo-equivalent model is generated by using a group of 5 different natural chemicals (M5: IL-17A, IL-22, Oncostatin-M, IL-1α, and TNF-α) on human epidermal cells keratinocytes, recapitulating the in vivo immune dysregulation as identified in vivo in the skin lesions of patients affected by psoriasis (Guilloteau et al., Skin Inflammation Induced by the Synergistic Action of IL-17A, IL-22, Oncostatin M, IL-1α, and TNF-α Recapitulates Some Features of Psoriasis. Journal of Immunology 2010; 184:5263-70). Thus, this in vivo-equivalent model provides an excellent tool for the investigation of the combined effect of anti-TNF-α and diacerein on psoriasis.

The experiments were carried out in the following manner: Primary human keratinocytes (purchased from Invitrogen, Carlsbad, Calif.) Epilife culture medium supplemented with human keratinocyte growth supplement (Invitrogen) in standard humidified chamber (37° C., 5% CO2). When the cells reached 80% confluency, cells were divided into 5 groups for treatment:
1). Cell culture medium alone, serving as a negative control
2). Cell culture medium plus 5 natural chemicals (M5)
3). Cell culture medium plus 5 natural chemicals (M5) and anti-TNF-alpha (infliximab)
4). Cell culture medium plus 5 natural chemicals (M5) and diacerein
5). Cell culture medium plus 5 natural chemicals (M5), anti-TNF-alpha (infliximab) and diacerein.

The above 5 groups were incubated at 37° C. for 24 hours.

The concentrations and commercial sources of the experiment components are:
IL-17 alpha (10 ng/ml, Life Technologies, Frederick, Md.)
IL-22 (10 ng/ml. Life Technologies, Frederick, Md.)
Oncostatin-M (10 ng/ml, Life Technologies, Frederick, Md.)
IL-1 alpha (10 ng/ml, Life Technologies, Frederick, Md.)
TNF-alpha (long/ml, Life Technologies, Frederick, Md.)
Diacerein (10 μM, Sigma Aldrich, Saint Louis, Md.)
Infliximab (Remicade®, an anti-TNF-alpha, 5 μg/ml, Jansen Biotech, Horsham, Pa.)

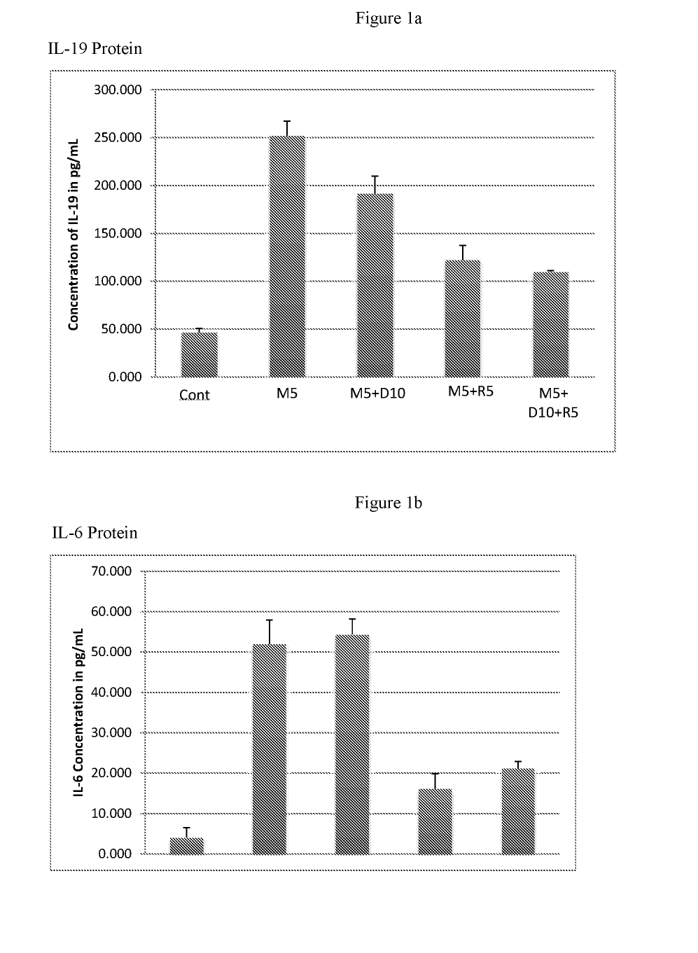

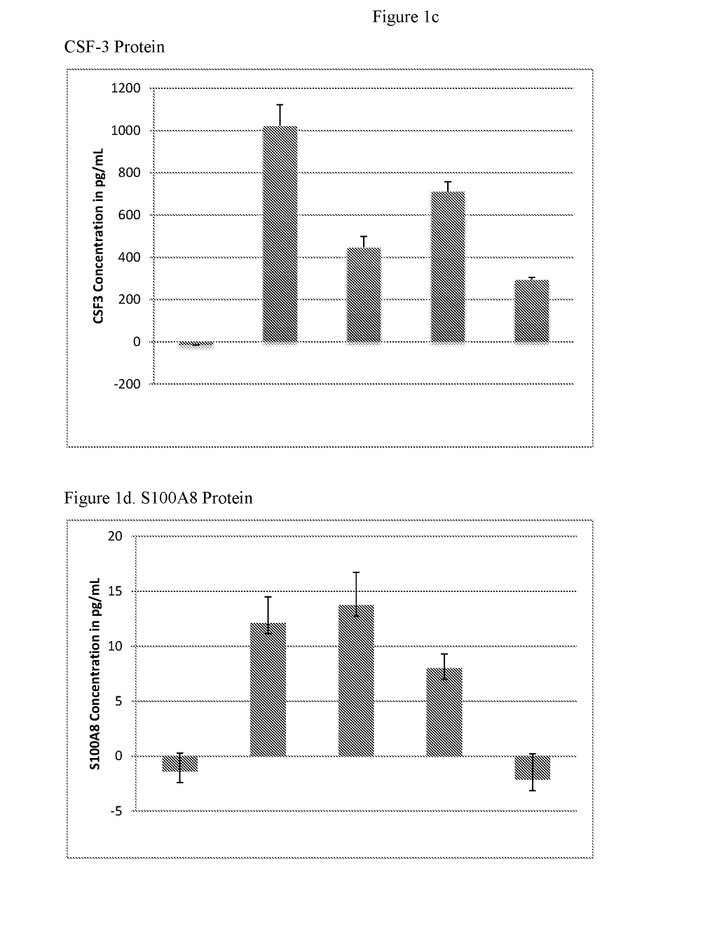

After 24 hours of incubation, the culture media (supernatant) of the above 5 groups were collected and stored at −20° C. for ELISA experiments as the following: 50-100 μl of cell culture supernatant were assayed with commercially available ELISA kits for human cytokines according to the manufacturers' instructions (IL-6, IL-19, G-CSF, S100AB, and NAP-2). The data we collected determined that infliximab alone (group 3), diacerein alone (group 4), and combined infliximab and diacerein (group 5) partially or completely reverse the dysregulation effect of the 5 natural chemicals (M5) in five, three, and five genes, respectively. The combined infliximab and diacerein has the greatest reversal effects in four of these five gene products, namely IL-19, G-CSF, S100AB, and NAP-2 as shown in FIGS. 1a-1e.

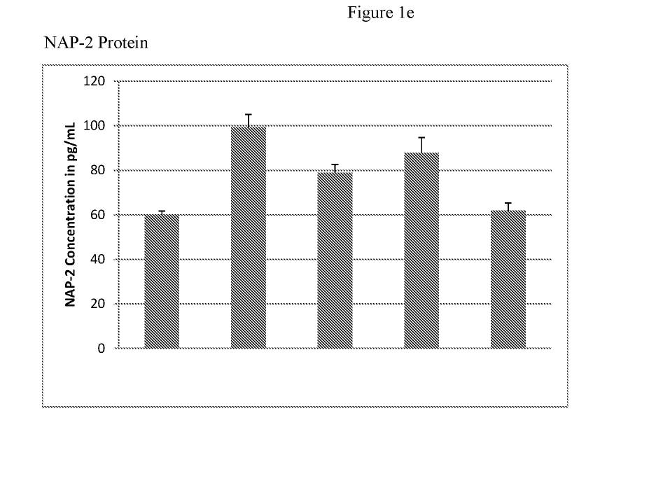

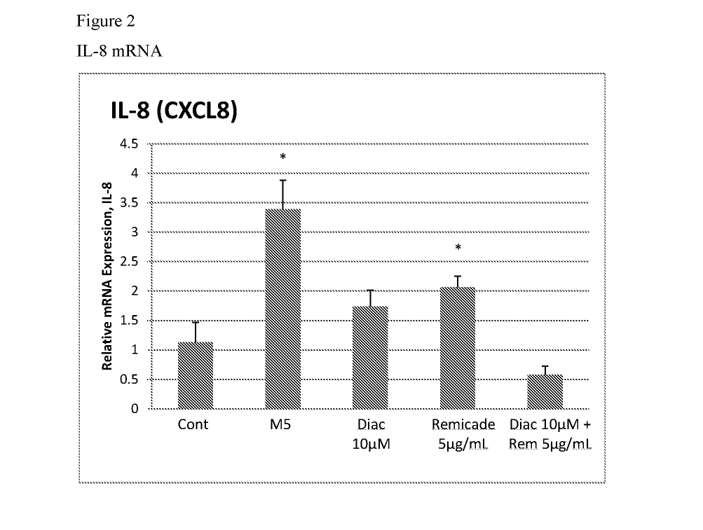

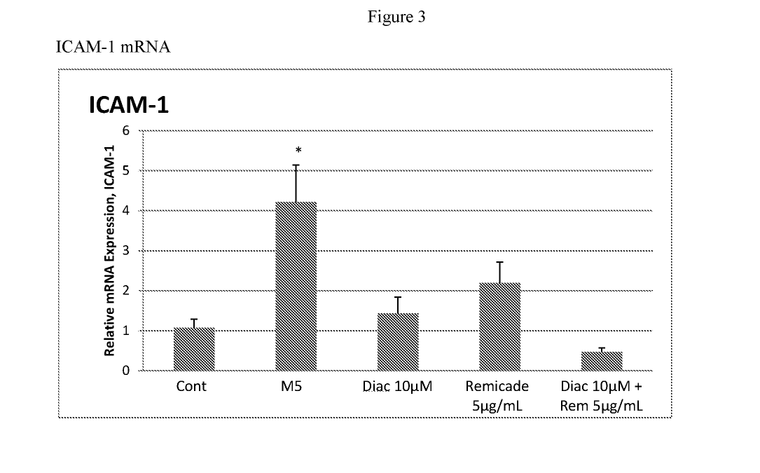

After 24 hours of incubation, the cultured cells were washed three times with sterile PBS (phosphate-buffered saline) and the RNAs were harvested with Trizol (Invitrogen) and the purity (A260/A280 ratio>1.8) was confirmed. Reverse transcription was performed with 1 micro gram RNA using first strand kit from Invitrogen according to manufacturer's instruction. 10 μl of mastermix (Qiagen, Valencia, Calif.) with the reversely transcribed cDNA were amplified using a real-time PCR machine (ViiA7 PCR, Applied Biosystems, Waltham, Pa.). Statistical analysis was performed with web-based Software. The experiment data determined that the combined action of anti-TNF alpha and diacerein were the most effective in reversing the M5-induced upregulation of two pro-inflammatory cytokine genes, namely interleukin-8 (IL-8) and intercellular adhesion molecule-1 (ICAM-1). See FIGS. 2-3.

In addition, the RNAs harvested as depicted in the above paragraph were subjected to cDNA microarray assay in order to examine a broader effect of diacerein on multiple pro-inflammatory cytokines upregulated by M5, using an Inflammatory Response and Autoimmune PCR Array (Qiagen) Statistical analysis was performed with a web-based Array Analysis Software (Qiagen). The results identified 81 pro-inflammatory genes significantly (p<0.05) regulated at least 2-fold by M5 compared to control. Diacerein alone, infliximab alone, and diacerein and infliximab together partially or fully reversed the effect of the five cytokines on 61, 67, and 71 genes respectively. Of 48 genes partially or fully reversed by all three groups, the greatest effect was seen with diacerein alone in 17 genes, infliximab alone in 10 genes, and diacerein and infliximab together in 21 genes. Thus the combined diacerein and infliximab has greater anti-inflammatory effects on higher of genes than either diacerein or infliximab alone.

Accordingly, as shown by this example, the combination of infliximab and diacerein provides a superior and synergistic therapeutic option for chronic inflammatory disease, such as psoriasis.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A method of treating a subject suffering from a inflammatory disease, the method comprising the steps of:
administering to the subject in need of treatment thereof a therapeutically effective amount of at least one anti-TNF alpha inhibitor and a therapeutically effective amount diacerein.

2. The method of claim 1, wherein the anti-TNF alpha inhibitor infliximab, adalimumab, certolizumab, etanercept, golimumab or combinations thereof.

3. The method of claim 2, wherein the anti-TNF alpha inhibitor is infliximab.

4. The method of claim 1, wherein the anti-TNF alpha inhibitor and diacerein are administered simultaneously.

5. The method of claim 1, wherein the anti-TNF alpha inhibitor and diacerin are administered sequentially.

6. The method of claim 1, wherein the amount of anti-TNF alpha inhibitor administered to the subject is about 0.25 mg/kg to about 10 mg/kg.

7. The method of claim 1, wherein the amount of diacerein administered to the subject is about 10 mg, 25 mg, 50 mg, or 75 mg.

8. The method of claim 7, wherein the diacerein is administered to the subject once or twice each day.

9. The method of claim 1, wherein the inflammatory disease is an acute inflammatory disease or chronic inflammatory disease.

10. The method of claim 9, wherein the inflammatory disease is chronic inflammatory disease.

11. The method of claim 10, wherein the chronic inflammatory disease is rheumatoid arthritis, psoriasis, ulcerative colitis, Crohn's disease, ankylosing spindilitis, psoriatic arthritis, non-infectious uveitis, hidradenitis suppurativa or lichen planus.
